(19) United States
(12) Patent Application Publication
(10) Pub. No.: US 2022/0031208 A1
(43) Pub. Date: Feb. 3, 2022

(54) MACHINE LEARNING TRAINING FOR MEDICAL MONITORING SYSTEMS

(71) Applicant: Covidien LP, Mansfield, MA (US)

(72) Inventors: Dean Montgomery, Edinburgh (GB); Paul S. Addison, Scotland (GB)

(21) Appl. No.: 16/942,034

(22) Filed: Jul. 29, 2020

(51) Int. Cl. A61B 5/1455; G06N 20/00; A61B 5/00; G06N 3/04

(52) U.S. Cl. CPC A61B 5/14552; G06N 3/04; A61B 5/746; G06N 20/00

(57) ABSTRACT

The present technology relates to the field of medical monitoring systems. Systems, methods, and computer readable media are described. In some embodiments, a truth data set and a sensor data set are accessed. The truth data set is associated with a plurality of test data acquired through a series of tests. The sensor data set is associated with a plurality of sensor data acquired from a medical monitoring device. A machine learning network associated with a medical monitoring system is trained based on the truth data set and the sensor data set.

### FIELD

The present technology is generally related to medical monitoring systems, and more particularly to machine learning training for medical monitoring systems.

### BACKGROUND

Many conventional medical monitors require attachment of a sensor to a patient in order to detect physiologic signals from the patient. These monitors process the received signals and determine vital signs, such as the patient's pulse rate, respiration rate, and oxygen saturation. For example, a pulse oximeter can be a finger sensor that includes two light emitters and a photodetector. The sensor emits light into the patient's finger and transmits the detected light signal to a monitor. The monitor can process the signal, determine vital signs (e.g., pulse rate, respiration rate, oxygen saturation), and display the vital signs on a display. Other pulse oximeter sensor variations can include forehead pulse oximeter sensors, adhesive pulse oximeter sensors, and non-adhesive pulse oximeter sensors configured to be held in contact with a body part of a patient.

Medical monitor sensors may be sensitive to patient movement. Such medical monitor sensors are typically calibrated to nominal conditions of a patient statically positioned. Further, physical characteristics of patients can vary along with environmental conditions, such as ambient lighting, which may impact sensed data characteristics.

### SUMMARY

The techniques of this disclosure generally relate to a machine learning training for medical monitoring systems.

In one aspect, a system includes a processing system and a memory system in communication with the processing system. The memory system can store instructions that when executed by the processing system result in accessing a truth data set and a sensor data set. The truth data set is associated with a plurality of test data acquired through a series of tests. The sensor data set is associated with a plurality of sensor data acquired from a medical monitoring device. A machine learning network associated with a medical monitoring system is trained based on the truth data set and the sensor data set.

In another aspect, a method includes accessing, by a processing system, a truth data set associated with a plurality of test data acquired through a series of tests. The processing system accesses a sensor data set associated with a plurality of sensor data acquired from a medical monitoring device. The processing system can train a machine learning network associated with a medical monitoring system based on the truth data set and the sensor data set.

In a further aspect, a computer program product includes a storage medium embodied with computer program instructions that when executed by a computer cause the computer to implement accessing a truth data set and a sensor data set. The truth data set is associated with a plurality of test data acquired through a series of tests. The sensor data set is associated with a plurality of sensor data acquired from a medical monitoring device. A machine learning network associated with a medical monitoring system is trained based on the truth data set and the sensor data set.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure. The drawings should not be taken to limit the disclosure to the specific embodiments depicted, but are for explanation and understanding only.

FIG. 1 is a block diagram of a medical monitoring system, in accordance with various embodiments of the present technology;

FIG. 2 is a block diagram of a system, configured in accordance with various embodiments of the present technology;

FIG. 3 is a flow diagram of training a machine learning network for a medical monitoring system configured in accordance with various embodiments of the present technology;

FIG. 4 is a block diagram illustrating a machine learning network configured in accordance with various embodiments of the present technology;

FIG. 5 is a flow diagram illustrating training of a machine learning network for a medical monitoring system configured in accordance with various embodiments of the present technology;

FIG. 6 is a flow diagram illustrating training of a machine learning network for a medical monitoring system configured in accordance with various embodiments of the present technology;

FIG. 7 is a flow diagram illustrating training of a machine learning network for a medical monitoring system configured in accordance with various embodiments of the present technology;

FIG. 8 is a plot of an adjustment to blood oxygen saturation as a percentage versus time in accordance with various embodiments of the present technology; and

FIG. 9 is a plot of an adjustment to blood oxygen saturation as a percentage versus time in accordance with various embodiments of the present technology.

### DETAILED DESCRIPTION

The following disclosure describes patient monitoring devices, systems, and associated methods for detecting and/or monitoring one or more patient parameters, such as oxygen saturation, heart rate, and/or others. As described in greater detail below, devices, systems, and/or methods configured in accordance with embodiments of the present technology are configured to train a machine learning network for a medical monitoring system. A medical monitoring system, such as a pulse oximeter, can use one or more calibration curves to correct for errors. Calibration curves can be generated using a test pulse oximeter attached to a volunteer. The volunteer can change breathing patterns or be provided with lower levels of oxygen while data from the test pulse oximeter is collected. Several blood draws can be performed and examined as co-oximeter data. Co-oximeter data collection is a slow process but has a high level of accuracy. By comparing co-oximeter data to pulse oximeter data collected at the same time with the same conditions, differences in results can be observed as error data. Due to the limited number of data points as constrained by blood draw intervals, error relationships can be generalized as calibration graphs which may assume a substantially linear relationship between collected data points. This approach can work well for many scenarios but may not work as well under other conditions, such as when a patient is moving.

Embodiments use a machine learning network to establish learned relationships between inputs to a medical monitoring system and outputs. Rather than using a conversion equation or calibration adjustments in combination with a conversion equation, the machine learning network learns many relationships that may not be readily apparent to a human observer. A trained machined learning network can improve result accuracy, particularly for conditions that are not well quantified through a conversion equation even when calibration is used. In order to get higher accuracy results, the machine learning network can be trained as further described herein.

Additionally, devices, systems, and/or methods configured in accordance with embodiments of the present technology can include one or more sensors or probes associated with (e.g., contacting) a patient that can be configured to capture data (e.g., oxygen saturation, temperature, blood pressure, heart rate, etc.) related to a patient. The devices, systems, and/or methods can transmit the captured data to a monitoring device, hub, mobile patient management system (MPM), or the like. In some embodiments, the devices, systems, and/or methods can analyze the captured data to determine and/or monitor one or more patient parameters using a machine learning network to determine a physiologic state of the patient. In these and other embodiments, the devices, systems, and/or methods in a test environment capture data using one or more sensors or probes to establish a training data set for the machine learning network. In conjunction with capturing sensor data, a second method of data collection can be used, such as blood draw testing, to establish a truth data set under substantially similar conditions as the sensor data is collected. The truth data and sensor data can be collectively used to train the machine learning network.

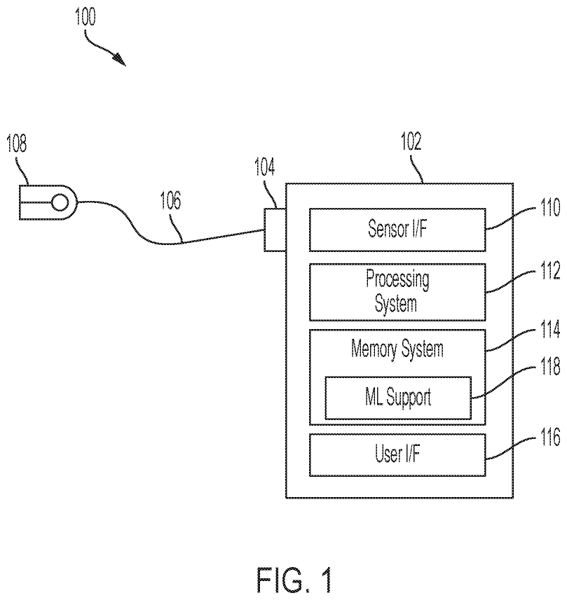

Specific details of several embodiments of the present technology are described herein with reference to FIGS. 1-9. Although many of the embodiments are described with respect to devices, systems, and methods for machine learning training for medical systems, other applications and other embodiments in addition to those described herein are within the scope of the present technology. For example, at least some embodiments of the present technology can be useful for detection and/or monitoring of one or more parameters of other animals and/or in non-patients (e.g., elderly or neonatal individuals within their homes, individuals in a search and rescue or stranded context, etc.). It should be noted that other embodiments in addition to those disclosed herein are within the scope of the present technology. Further, embodiments of the present technology can have different configurations, components, and/or procedures than those shown or described herein. Moreover, a person of ordinary skill in the art will understand that embodiments of the present technology can have configurations, components, and/or procedures in addition to those shown or described herein and that these and other embodiments can be without several of the configurations, components, and/or procedures shown or described herein without deviating from the present technology.

FIG. 1 is a block diagram of an exemplary monitoring system 100 that includes a patient monitoring device 102 operably coupled through a connector 104 and transmission path 106 to a sensor 108. The connector 104 may support wired, wireless, optical, or magnetic communication through the transmission path 106 to the sensor 108. In the example of FIG. 1, the sensor 108 can be a pulse oximetry sensor configured to emit and detect a red light signal and an infrared light signal as photoplethysmographic (PPG) signals. Detected light signals at the sensor 108 can be communicated through the transmission path 106 and connector 104 to a sensor interface 110 of the patient monitoring device 102, for instance, as a red signal input and an infrared signal input. The red signal input and infrared signal input may be electrically encoded signals at the sensor 108 and transmitted as analog or digitally sampled signals. The patient monitoring device 102 can also include a processing system 112, a memory system 114, a user interface 116, and/or other elements (not depicted). The memory system 114 can store sampled values of the red signal input and infrared signal input used to determine, for instance, a blood oxygen saturation as a percentage (SpO₂) and various derived signals indicative of a condition of a patient using the sensor 108. Rather than using an equation-based computation to determine SpO₂, embodiments can use a machine learning network based on executable instructions and data structures as machine learning support 118. The machine learning support 118 can be stored in the memory system 114. In some embodiments, the processing system 112 may have hardware that further accelerates machine learning performance, such as architected processing units to compute weights and node values in parallel, for instance.

The user interface 116 can be a monitor with a screen (e.g., to display various information, such as a power on/off button, one or more patient parameters, one or more alerts and/or alarms, etc.). The patient monitoring device 102 can be attached to, be worn, and/or otherwise be carried by a patient. For example, the patient monitoring device 102 and the sensor 108 can be attached to and/or worn by the patient. In some embodiments, the patient monitoring device 102 can be sewn into the patient's clothing. In these and other embodiments, the patient monitoring device 102 can be a mobile device, such as a mobile phone, tablet, or laptop.

In the embodiments illustrated in FIG. 1, the sensor 108 can include a pulse oximeter attachable to a finger of the patient. In these and other embodiments, other sensors in addition to or in lieu of the pulse oximeter can be used, such as electrodes, temperature sensors, blood pressure cuffs, etc. The sensor 108 and one or more other sensors can be used to perform various tests and/or to capture various information and data relating to the patient. For example, the sensor 108 can be used in combination with capturing an electrocardiogram (ECG) signal and/or an electroencephalogram (EEG) signal of the patient. In these and other embodiments, the one or more sensors can capture the patient's oxygen saturation, temperature, blood pressure, and/or other patient parameters (e.g., systolic and diastolic pressure, heart rate, respiratory rate, average temperature, etc.). Additionally or alternatively, the sensor 108 and/or the patient monitoring device 102 can transmit captured information for further processing via one or more wired and/or wireless connections.

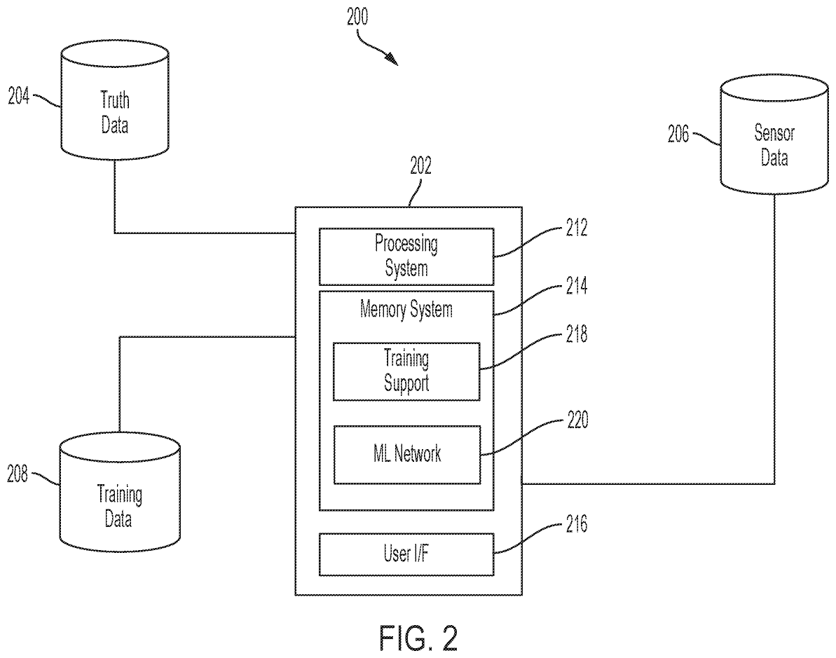

FIG. 2 depicts an example of a system 200 for training a machine learning network that can be used as part of the machine learning support 118 of FIG. 1. A computer system 202 of the system 200 can include a processing system 212, a memory system 214, a user interface 216, and/or other elements (not depicted). The computer system 202 can be any type of computer known in the art, such as a server, a personal computer, a laptop computer, a cloud computing resource, a tablet computer, or a wearable computer. Further, the computer system 202 can be distributed between multiple computing and storage devices. The user interface 216 can include any type of user input/output interface that enables a user to access the computer system 202, such as a keyboard, mouse, touchscreen, video display, and the like. User interface devices can be located remotely and connected to the user interface 216 through a wired, wireless, and/or network interface.

In the example of FIG. 2, the computer system 202 can access truth data 204 and sensor data 206, which may be stored locally or remotely with respect to the computer system 202. The computer system 202 can generate and update training data 208 using training support 218 to train a machine learning network 220. The training support 218 may be embodied in instructions stored in the memory system 214 and executable by the processing system 212. The machine learning network 220 can be a network of nodes as part of a data structure in the memory system 214, where the training support 218 can tune weights of the machine learning network 220 as relationships are learned with respect to the truth data 204, sensor data 206, and the training data 208.

The computer system 202 can be operated in a testing and development environment to train the machine learning network 220 with training results used to configure the machine learning support 118 of FIG. 1. In the example of FIG. 2, truth data 204 and/or the sensor data 206 can be collected as part of calibration data collection process. As one example, a volunteer can be connected to the sensor 108 of the medical monitoring system 100 of FIG. 1 with machine learning support 118 disabled or operated in a training mode. As oxygen levels are established and adjusted for the volunteer, data from the sensor 108 can be captured as sensor data 206. At blood draw intervals, blood can be drawn from the volunteer for co-oximeter analysis. Results of the co-oximeter analysis can be stored in the truth data 204. The number of data values collected in the sensor data 206 can greatly exceed the number of data values collected in the truth data 204. The training support 218 can use the combination of the truth data 204 and sensor data 206 to produce the training data 208 and subsequently train the machine learning network 220 based on the training data 208. The training data 208 can be populated incrementally. For example, the training data 208 may initially include values and labels based on the sensor data 206, which are then further blended or weighted based on the truth data 204. Alternatively, the training data 208 can be populated by selecting one or more portions of the sensor data 206 and forming labeled training data values as an aggregate set with the truth data 204. Other options and variations are further described herein and may be combined, extended, or simplified depending upon the amount of data, processing resources, and desired accuracy.

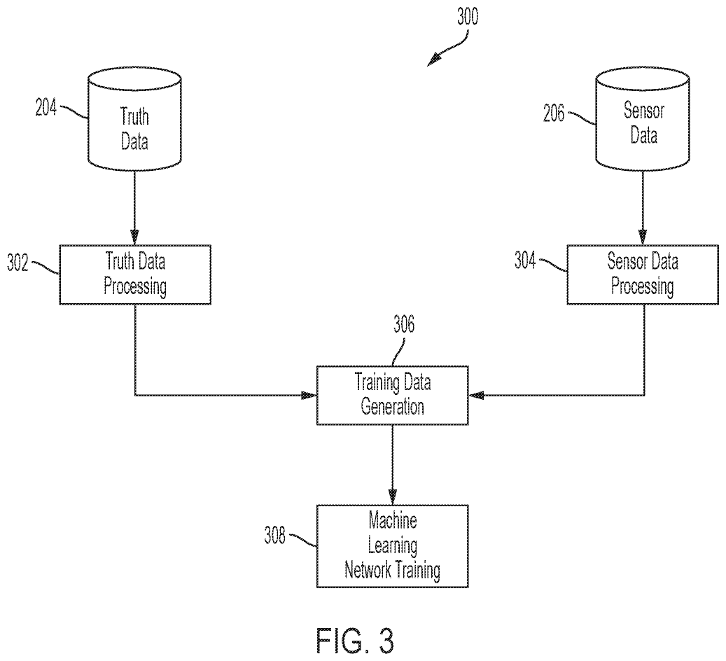

FIG. 3 is a flow diagram of a process 300 for training the machine learning network 220 of FIG. 2 for a medical monitoring system, configured in accordance with various embodiments of the present technology. The process 300 can be used to train the machine learning network 220 of FIG. 2 associated with a medical monitoring system based on a truth data set of truth data 204 and a sensor data set of sensor data 206. In process 300, truth data processing 302 can access the truth data set associated with a plurality of test data acquired through a series of tests, such as blood draws of a volunteer receiving different levels of oxygen. Sensor data processing 304 can access the sensor data set associated with a plurality of sensor data acquired from a medical monitoring device, such as an instance of the medical monitoring system 100 of FIG. 1 used for collecting data through sensor 108. The number of data values in the sensor data set can be greater than the number of data values in the truth data set.

The sensor data 206 can include raw values of the red signal input and infrared signal input from sensor 108. Alternatively, the sensor data 206 may be stored as intermediate values, such as a ratio of ratios. A ratio of ratios can be a red ratio divided by an infrared ratio, where the red ratio is a ratio of detected red light alternating current to detected red light direct current, and the infrared ratio is a ratio of detected infrared light alternating current to detected infrared light direct current. Further, the sensor data 206 be in a processed form as sensed SpO₂.

Output of the truth data processing 302 and the sensor data processing 304 can be provided to training data generation 306. The training data generation 306 can determine how to combine processed values from the truth data processing 302 based on truth data 204 and from the sensor data processing 304 based on sensor data 206. The training data generation 306 can output the training data 208 of FIG. 2. Machine learning network training 308 can use the training data 208 to train the machine learning network 220 of FIG. 2. The truth data processing 302, sensor data processing 304, training data generation 306, and machine learning network training 308 can be subcomponents of the training support 218 of FIG. 2.

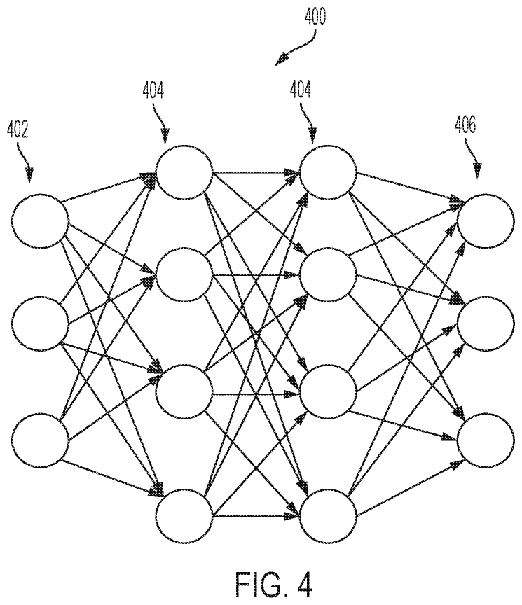

FIG. 4 is a block diagram illustrating a machine learning network 400 configured in accordance with various embodiments of the present technology. The machine learning network 400 is an example of the machine learning network 220 of FIG. 2. The machine learning network 400 can include a plurality of input nodes 402, intermediate layer nodes 404, and output nodes 406. The machine learning network 400 can be a deep learning neural network, for example. The intermediate layer nodes 404 can be organized in one or more hidden layers between the input nodes 402 and the output nodes 406. The intermediate layer nodes 404 can support modeling of complex non-linear relationships between the input nodes 402 and output nodes 406. Each of the intermediate layer nodes 404 can have associated weights with connections defined in a feedforward direction. Alternatively, the machine learning network 400 can be configured as a recurrent neural network supporting data flow in any direction and may use long short-term memory (LSTM). Other example configurations can include a convolutional neural network (CNN), which may be a Resnet style network or other CNN with at least one regression output layer. Operational sequences for processing the machine learning network 400 can include known techniques, such as max-pooling operations, rectifying, smoothing, dropout, convolution, normalization, addition, regression, and the like with various learning approaches to train the machine learning network 400 to establish weights.

Training of the machine learning network 400 can use supervised learning or semi-supervised learning. More complex configurations can require a relatively large training data set size to converge on a set of weights through multiple training iterations. Training can use a combination of labeled and unlabeled data. Labeling of training data can assist the training process complete faster with fewer total samples needed. As part of training, a training data set containing many examples can be passed input into the machine learning network 400, and the machine learning network 400 can output results with confidence values indicating how well the input matches particular patterns. Results with confidence levels above a confidence threshold can be classified as most likely correct. Results with confidence levels below the confidence threshold may be classified as indeterminant. Although the example of FIG. 4 depicts three input nodes 402 and three output nodes 406, it will be understood that any number of input nodes 402 and output nodes 406 can be used depending upon the desired machine learning approach. For example, the input nodes 402 may receive SpO₂ data, ratio data, co-oximeter data, red signal input, infrared signal input, derived data, and/or other sensor data. In some embodiments, the co-oximeter data is used primarily to establish labels or to adjust other data values. As an alternative, where a sufficient number of co-oximeter data values are available and other data is used for labeling, the co-oximeter data may be used as an input. The output nodes 406 may indicate SpO₂ value ranges, ratio data ranges, and/or various predictions/classifications with associated levels of confidence.

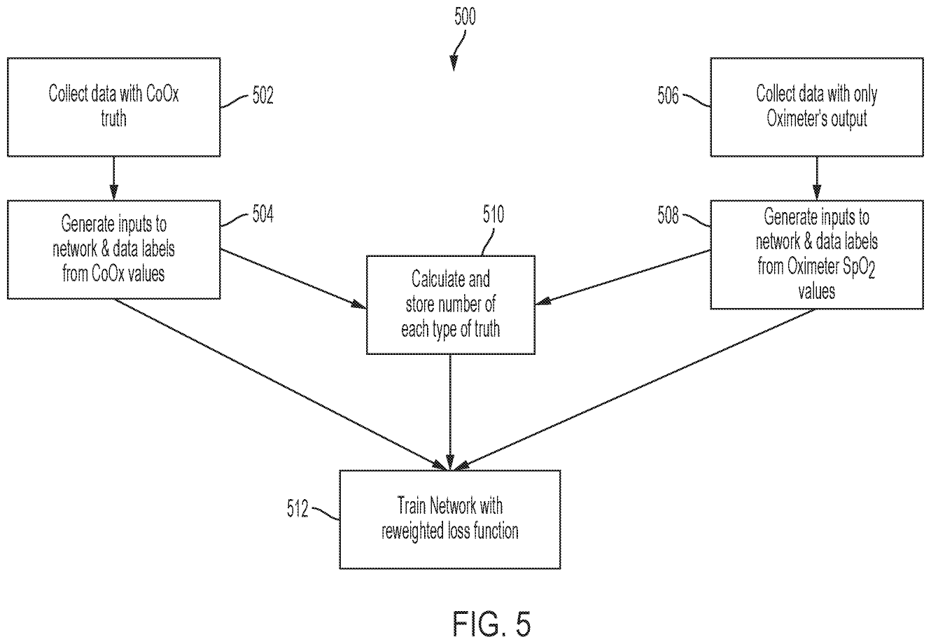

FIG. 5 is a flow diagram illustrating a process 500 to train a machine learning network for a medical monitoring system in accordance with various embodiments of the present technology. The process 500 can be incorporated in the training support 218 of FIG. 2. The process 500 is described with respect to the system 200 of FIG. 2 but may be used in a variety of system configurations.

The process 500 can begin at block 502 by accessing or collecting data with a co-oximeter truth, such as accessing the truth data 204. At block 504, a first plurality of inputs to the machine learning network 220 and data labels are generated based on the truth data 204. At block 506, data can be accessed or collected that include oximeter output, for instance, by accessing the sensor data 206. At block 508, a second plurality of inputs to the machine learning network 220 and data labels are generated based on the sensor data 206. At block 510, a plurality of training data 208 is populated based on a combination of the first plurality of inputs with data labels and the second plurality of inputs with data labels. Populating the training data 208 can include calculating and storing a number of each type of “truth” based on the first and second inputs.

In embodiments, the sensor data 206 can be used for data labels in the training data 208 for values where there is no corresponding value in the truth data 204. Where truth data 204 is available, the training data 208 may be labeled based on the truth data 204. Since this may lead to a relatively small number of values in the training data 208 labeled based on the truth data 204, other adjustments can be made to blend or mix values from the sensor data 206 with the truth data 204 to populate the training data 208. As one example, a loss function used during training can be weighted to give a greater weight to contributions of the truth data 204. For instance, a loss function may be of the form: L_{weighted}=w₁*L_{COOX}+w₂*L_{PulseOx}, where w₁ and w₂ are precalculated to tune the contribution from each data source with L_{COOX} based on the truth data 204 and L_{PulseOx} based on the sensor data 206. To evenly balance the contributions, weighting functions may be defined as w₂=N_{COOX}/N_{TOTAL}, w₁=N_{PulseOx}/N_{TOTAL}. Here, N_{COOX} can be the number of values used from the truth data 204, N_{PulseOx} can be the number of values used from the sensor data 206, and N_{TOTAL} can be the total number of values used. As another example, to over represent the number of data points from the truth data 204, the weighting functions may be defined as: w₂=2*N_{COOX}/N_{TOTAL}, w₂=N_{PulseOx}/N_{TOTAL}. Other variations are contemplated. At block 512, training of the machine learning network 220 can be performed based on the training data 208, and a loss function applied to the combination of the first plurality of inputs with data labels and the second plurality of inputs with data labels. The loss function can be applied to adjust a relative weight of data from the truth data 204 with respect to the sensor data 206. Weighting of the loss function can be adjusted as described above or using other techniques and/or relative weighting values.

Although the steps of the process 500 are discussed and illustrated in a particular order, the process 500 illustrated in FIG. 5 is not so limited. In other embodiments, the process 500 can be performed in a different order. In these and other embodiments, any of the steps of the process 500 can be performed before, during, and/or after any of the other steps of the process 500. A person of ordinary skill in the relevant art will readily recognize that the illustrated method can be altered and still remain within these and other embodiments of the present technology.

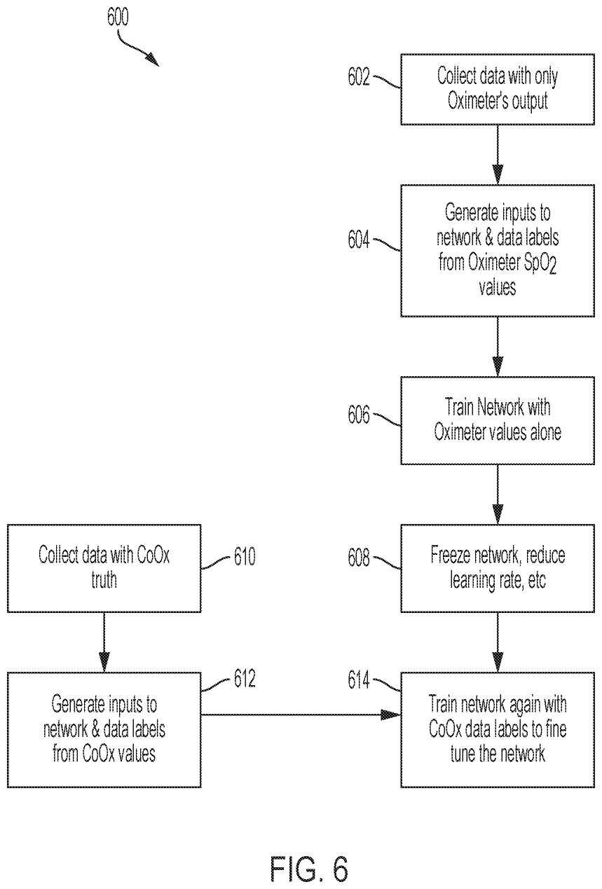

FIG. 6 is a flow diagram illustrating a process 600 to train a machine learning network for a medical monitoring system in accordance with various embodiments of the present technology. The process 600 can be incorporated in the training support 218 of FIG. 2. The process 600 is described with respect to the system 200 of FIG. 2 but may be used in a variety of system configurations.

The process 600 can begin at block 602 by accessing or collecting data values that include oximeter output, for instance, by accessing the sensor data 206. At block 604, a first plurality of inputs to the machine learning network 220 and data labels are generated based on the sensor data 206. At block 606, the machine learning network 220 can be initially trained based on the inputs and labels from the sensor data 206. At block 608, the machine learning network 220 can be frozen by holding weights in some layers, or a learning rate can be reduced in subsequent training to retain a portion of the initial learning based on the sensor data 206 for transfer learning. Transfer learning can retain a portion of initial learning while refining other weights based on different or adjusted training data.

At block 610, data can be accessed or collected that include a co-oximeter truth, such as accessing the truth data 204. At block 612, a second plurality of inputs to the machine learning network 220 and data labels are generated based on the truth data 204. At block 614, transfer learning training of the machine learning network 220 can be performed based on truth data 204 as higher quality fine tuning data. This approach can retrain the machine learning network 220 based on the second plurality of inputs with data labels to fine tune initial training performed with respect to the first plurality of inputs with data labels. Other transfer learning approach variations are contemplated. For example, initial weights can be based on data from different sensor versions or data from a different collection of volunteers, with further refinements based on an updated/new data set.

Although the steps of the process 600 are discussed and illustrated in a particular order, the process 600 illustrated in FIG. 6 is not so limited. In other embodiments, the process 600 can be performed in a different order. In these and other embodiments, any of the steps of the process 600 can be performed before, during, and/or after any of the other steps of the process 600. A person of ordinary skill in the relevant art will readily recognize that the illustrated method can be altered and still remain within these and other embodiments of the present technology.

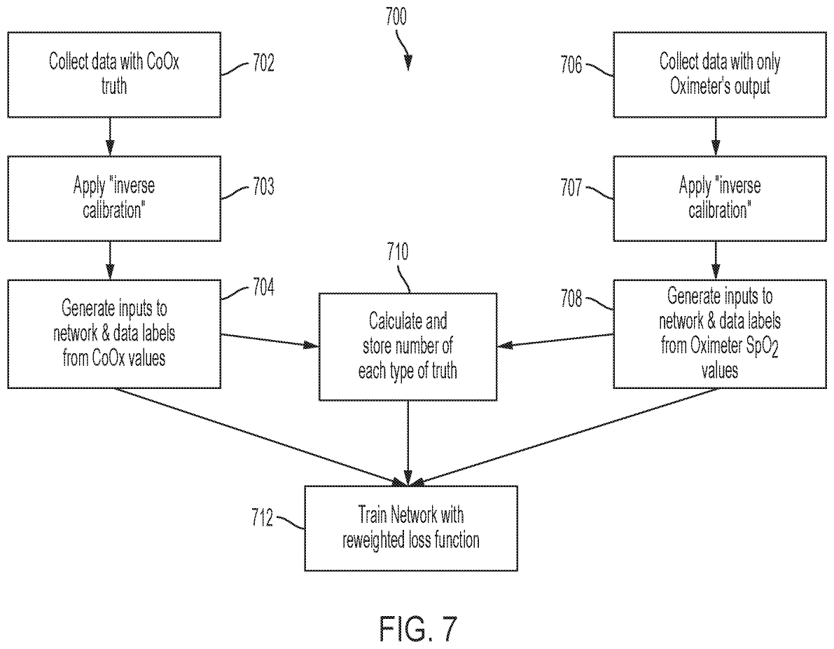

FIG. 7 is a flow diagram illustrating a process 700 to train a machine learning network for a medical monitoring system in accordance with various embodiments of the present technology. The process 700 can be incorporated in the training support 218 of FIG. 2. The process 700 is described with respect to the system 200 of FIG. 2 but may be used in a variety of system configurations.

The process 700 can begin at block 702 by accessing or collecting data with a co-oximeter truth, such as accessing the truth data 204. At block 704, a first plurality of inputs to the machine learning network 220 and data labels are generated based on the truth data 204. Where the truth data 204 is stored in a different format than the data format expected by the machine learning network 220, an inverse calibration or format conversion can be applied in block 703. For example, if the truth data 204 includes SpO₂ values computed based a blood draw, block 703 may compute sensor data values that would result in equivalent SpO₂ values as an inverse calibration. Other examples can include determining an equivalent ratio-of-ratios value or other such data formats for use in training the machine learning network 220.

At block 706, data can be accessed or collected that include oximeter output, for instance, by accessing the sensor data 206. At block 708, a second plurality of inputs to the machine learning network 220 and data labels are generated based on the sensor data 206. Where the sensor data 206 is stored in a different format than the data format expected by the machine learning network 220, an inverse calibration or format conversion can be applied in block 707. For example, if the sensor data 206 includes SpO₂ values collected during testing of the patient monitoring device 102 of FIG. 1, block 707 may compute sensor data values that would result in equivalent SpO₂ values as an inverse calibration. Other examples can include determining an equivalent ratio-of-ratios value or other such data formats for use in training the machine learning network 220. The data format output of blocks 703 and 707 can be the same to support combining the data into the training data 208. Further, as part of block 707 or 708, preprocessing can be performed, such as that described in U.S. patent application Ser. No. 16/854,177, the disclosure of which is hereby incorporated herein by reference in its entirety. As an example, quality metrics may be stored with the sensor data 206 indicative of whether any issues were detected during data collection and used in subsequent analysis. For example, if there was signal noise above a noise threshold or other conditions that may reduce the quality of the data, the quality metrics can reflect such conditions. The quality metrics can be passed through as additional labels for training or can be used to select portions of the sensor data 206 to be discarded to avoid training with lower quality values, for instance, based on determining that one or more quality metrics are below a quality threshold. Further, preprocessing can include filtering to smooth data transients which may blend or exclude smaller scale transients and/or larger scale transients that are unlikely to reflect physiological activity.

At block 710, a plurality of training data 208 is populated based on a combination of the first plurality of inputs with data labels and the second plurality of inputs with data labels. Populating the training data 208 can include calculating and storing a number of each type of “truth” based on the first and second inputs. Block 710 can be substantially equivalent to block 510 as previously described with respect to FIG. 5. Similar to block 512 of FIG. 5 at block 712, training of the machine learning network 220 can be performed based on the training data 208, and a loss function for the combination of the first plurality of inputs with data labels and the second plurality of inputs with data labels can be applied to adjust a relative weight of data from the truth data 204 with respect to the sensor data 206.

Although the steps of the process 700 are discussed and illustrated in a particular order, the process 700 illustrated in FIG. 7 is not so limited. In other embodiments, the process 700 can be performed in a different order. In these and other embodiments, any of the steps of the process 700 can be performed before, during, and/or after any of the other steps of the process 700. A person of ordinary skill in the relevant art will readily recognize that the illustrated method can be altered and still remain within these and other embodiments of the present technology.

The processes 300, 500, 600, 700 of FIGS. 3 and 5-7 may be combined, performed in the alternative, performed and compared, or further subdivided. For example, block 302 of process 300 can represent a collection or generalization of processing performed on truth data 204 in processes 500, 600, and 700. Block 304 of process 300 can represent a collection or generalization of processing performed on sensor data 206 in processes 500, 600, and 700. Block 306 of process 300 can represent a collection or generalization of processing performed to produce or modify training data 208 in processes 500, 600, and 700. Block 308 of process 300 can represent a collection or generalization of training of the machine learning network 220 in processes 500, 600, and 700.

With respect to processes 500, 600, and 700, a set of training weights from process 500 can differ from training weights produced by processes 600 and 700. Further testing can be performed with respect to the machine learning network 220 using weights from each of the processes 500, 600, and 700 to determine which trained version of the machine learning network 220 has a higher accuracy. The best performing version of the machine learning network 220 can be used to program the machine learning support 118 of FIG. 1. Furthermore, as more test data and in-service data is collected over time, retraining or tuning of the machine learning network 220 can be performed. The machine learning network 220 can be configured to process various types of inputs and generate various types of outputs with respect to processes 500, 600, and 700. For example, the machine learning network 220 can be trained to predict a ratio of ratios based on a red signal input and an infrared signal input. The ratio of ratios can be converted to blood oxygen saturation as a percentage or other such values as a post-processing step. Further, the sensor data 206 can be used to generate one or more derived signals. For example, the one or more derived signals can include heart rate, pulse amplitude, high-pass filtered infrared/red, low-pass filtered infrared/red, skew of the pulses, area of the pulses, location of fiducial points (e.g., dichrotic notch location, peak location, secondary peak location, peak of the derivative, etc.) and/or other such signals. The one or more derived signals can be provided as input to the machine learning network 220. Other pre-processing and signal derivations are contemplated to further condition or tune the machine learning network 220.

The use of ratio-of-ratio values or derived signal values can support a wider range of sensors 108. For example, predicting ratio-of-ratios values by the machine learning support 118 of FIG. 1 can then allow for other localized calibration adjustments to be performed based on calibration characteristics associated with a specific version of the sensor 108 and/or patient monitoring device 102 of FIG. 1. The calibration adjustments may be used in the inverse transforms of block 703 and/or block 707, for example, to remove device specific adjustments during training of the machine learning network 220 depending on how the sensor data 206 is collected and formatted.

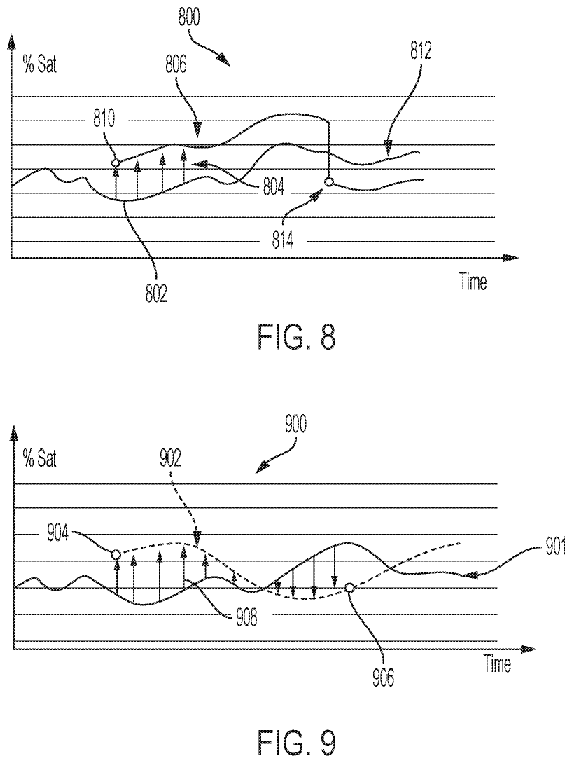

In various embodiments, combining of the truth data 204 with the sensor data 206 to produce training data 208 can include aggregation or biasing of data values. For example, at each blood draw a difference between an SpO₂ value of a pulse oximeter and the co-oximeter value can be used to calculate a bias. Between a blood draw and the next blood draw, the bias can be removed from the SpO₂ data labels. This can be visualized, for instance in FIG. 8 as a plot 800 of an adjustment to SpO₂ versus time. A difference 804 in SpO₂ can be computed based on a first portion 802 of a sensor data set and a truth data set based on observation of a modified SpO₂ value 806 at a blood draw 810. A second portion 812 of the sensor data set can be adjusted based on the difference observed at a subsequent blood draw 814. Transitions between the first portion 802 and the second portion 812 after difference adjustments can be further smoothed to reduce abrupt transitions.

FIG. 9 is a plot 900 of an adjustment to SpO₂ versus time in accordance with various embodiments of the present technology. In the example depicted in FIG. 9, linear interpolation of SpO₂ data labels 902 can be performed in between blood draws 904, 906 to improve the quality of a SpO₂ reference signal 901. This can result in a smoother transition between difference values 908 based on truth data of co-oximetry values.

In the examples of FIGS. 8 and 9, co-oximetry data values of truth data 204 of FIG. 2 may not be used directly during training of the machine learning network 220 of FIG. 2 but can be used to improve the quality of the values in the sensor data 206 of FIG. 2. In an alternate implementation, the approaches described in reference to FIGS. 8 and 9 may be combined with other approaches. For instance, the truth data 204 can be used to modify the sensor data 206, and the truth data 204 can be used during training of the machine learning network 220.

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, while steps are presented in a given order, alternative embodiments can perform steps in a different order. Furthermore, the various embodiments described herein can also be combined to provide further embodiments.

Instructions may be executed by one or more processors (e.g., processing systems 112, 212 of FIGS. 1 and 2), such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” or “processing system” as used herein may refer to any of the foregoing structure or any other physical structure suitable for implementation of the described techniques. Also, the techniques could be fully implemented in one or more circuits or logic elements.

The systems and methods described herein can be provided in the form of tangible and non-transitory machine-readable medium or media (such as a hard disk drive, hardware memory, etc.) having instructions recorded thereon for execution by a processor or computer. The set of instructions can include various commands that instruct the computer or processor to perform specific operations such as the methods and processes of the various embodiments described here. The set of instructions can be in the form of a software program or application as a computer program product. The computer storage media can include volatile and non-volatile media, and removable and non-removable media, for storage of information such as computer-readable instructions, data structures, program modules or other data. The computer storage media can include, but are not limited to, RAM, ROM, EPROM, EEPROM, flash memory or other solid-state memory technology, CD-ROM, DVD, or other optical storage, magnetic disk storage, or any other hardware medium which can be used to store desired information and that can be accessed by components of the system. Components of the system can communicate with each other via wired or wireless communication. The components can be separate from each other, or various combinations of components can be integrated together into a monitor or processor or contained within a workstation with standard computer hardware (for example, processors, circuitry, logic circuits, memory, and the like). The system can include processing devices such as microprocessors, microcontrollers, integrated circuits, control units, storage media, and other hardware.

From the foregoing, it will be appreciated that specific embodiments of the technology have been described herein for purposes of illustration, but well-known structures and functions have not been shown or described in detail to avoid unnecessarily obscuring the description of the embodiments of the technology. To the extent any materials incorporated herein by reference conflict with the present disclosure, the present disclosure controls. Where the context permits, singular or plural terms can also include the plural or singular term, respectively. Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Where the context permits, singular or plural terms can also include the plural or singular term, respectively. Additionally, the terms “comprising,” “including,” “having” and “with” are used throughout to mean including at least the recited feature(s) such that any greater number of the same feature and/or additional types of other features are not precluded.

From the foregoing, it will also be appreciated that various modifications can be made without deviating from the technology. For example, various components of the technology can be further divided into subcomponents, or various components and functions of the technology can be combined and/or integrated. Furthermore, although advantages associated with certain embodiments of the technology have been described in the context of those embodiments, other embodiments can also exhibit such advantages, and not all embodiments need necessarily exhibit such advantages to fall within the scope of the technology. Accordingly, the disclosure and associated technology can encompass other embodiments not expressly shown or described herein.

What is claimed is:

1. A system, comprising:
a processing system; and
a memory system in communication with the processing system, the memory system storing instructions that when executed by the processing system result in:
accessing a truth data set associated with a plurality of test data acquired through a series of tests;
accessing a sensor data set associated with a plurality of sensor data acquired from a medical monitoring device; and
training a machine learning network associated with a medical monitoring system based on the truth data set and the sensor data set.

2. The system of claim 1, wherein the test data comprises co-oximeter data, and the sensor data comprises pulse oximeter data.

3. The system of claim 1, wherein the machine learning network comprises a deep learning neural network.

4. The system of claim 1, further comprising instructions that when executed by the processing system result in:
generating a first plurality of inputs to the machine learning network and data labels based on the truth data set;
generating a second plurality of inputs to the machine learning network and data labels based on the sensor data set; and
populating a plurality of training data based on a combination of the first plurality of inputs with data labels and the second plurality of inputs with data labels, wherein training the machine learning network is performed based on the training data.

5. The system of claim 4, further comprising instructions that when executed by the processing system result in:
applying a loss function to the combination of the first plurality of inputs with data labels and the second plurality of inputs with data labels to adjust a relative weight of data from the truth data set with respect to the sensor data set.

6. The system of claim 4, further comprising instructions that when executed by the processing system result in:
applying an inverse calibration to either or both of data from the truth data set and the sensor data set, wherein the machine learning network is trained to predict a ratio of ratios based on a red signal input and an infrared signal input; and
converting the ratio of ratios to a blood oxygen saturation as a percentage.

7. The system of claim 1, further comprising instructions that when executed by the processing system result in:
generating a first plurality of inputs to the machine learning network and data labels based on the sensor data set;
training the machine learning network initially based on the first plurality of inputs with data labels; and
generating a second plurality of inputs to the machine learning network and data labels based on the truth data set, wherein training the machine learning network based on the truth data set and the sensor data set comprises retraining the machine learning network based on the second plurality of inputs with data labels to fine tune initial training performed with respect to the first plurality of inputs with data labels.

8. The system of claim 1, further comprising instructions that when executed by the processing system result in:
analyzing one or more quality metrics associated with the sensor data set; and
discarding a portion of the sensor data set based on determining that the one or more quality metrics are below a quality threshold.

9. The system of claim 1, further comprising instructions that when executed by the processing system result in:
generating one or more derived signals based on the sensor data; and
providing the one or more derived signals as input to the machine learning network.

10. The system of claim 1, further comprising instructions that when executed by the processing system result in:
determining a difference in a blood oxygen saturation as a percentage computed based on a first portion of the sensor data set and the truth data set; and
adjusting a second portion of the sensor data set based on the difference.

11. A method comprising:
accessing, by a processing system, a truth data set associated with a plurality of test data acquired through a series of tests;
accessing, by the processing system, a sensor data set associated with a plurality of sensor data acquired from a medical monitoring device; and
training, by the processing system, a machine learning network associated with a medical monitoring system based on the truth data set and the sensor data set.

12. The method of claim 11, wherein the test data comprises co-oximeter data, and the sensor data comprises pulse oximeter data.

13. The method of claim 11, wherein the machine learning network comprises a deep learning neural network.

14. The method of claim 11, further comprising:
generating a first plurality of inputs to the machine learning network and data labels based on the truth data set;
generating a second plurality of inputs to the machine learning network and data labels based on the sensor data set; and
populating a plurality of training data based on a combination of the first plurality of inputs with data labels and the second plurality of inputs with data labels, wherein training the machine learning network is performed based on the training data.

15. The method of claim 14, further comprising:
applying a loss function to the combination of the first plurality of inputs with data labels and the second plurality of inputs with data labels to adjust a relative weight of data from the truth data set with respect to the sensor data set.

16. The method of claim 14, further comprising:
applying an inverse calibration to either or both of data from the truth data set and the sensor data set, wherein the machine learning network is trained to predict a ratio of ratios based on a red signal input and an infrared signal input; and
converting the ratio of ratios to a blood oxygen saturation as a percentage.

17. The method of claim 11, further comprising:
generating a first plurality of inputs to the machine learning network and data labels based on the sensor data set;
training the machine learning network initially based on the first plurality of inputs with data labels; and
generating a second plurality of inputs to the machine learning network and data labels based on the truth data set, wherein training the machine learning network based on the truth data set and the sensor data set comprises retraining the machine learning network based on the second plurality of inputs with data labels to fine tune initial training performed with respect to the first plurality of inputs with data labels.

18. The method of claim 11, further comprising:
analyzing one or more quality metrics associated with the sensor data set; and
discarding a portion of the sensor data set based on determining that the one or more quality metrics are below a quality threshold.

19. The method of claim 11, further comprising:
generating one or more derived signals based on the sensor data; and
providing the one or more derived signals as input to the machine learning network.

20. The method of claim 11, further comprising:
determining a difference in a blood oxygen saturation as a percentage computed based on a first portion of the sensor data set and the truth data set; and
adjusting a second portion of the sensor data set based on the difference.

21. A computer program product comprising a storage medium embodied with computer program instructions that when executed by a computer cause the computer to implement:
accessing a truth data set associated with a plurality of test data acquired through a series of tests;
accessing a sensor data set associated with a plurality of sensor data acquired from a medical monitoring device; and
training a machine learning network associated with a medical monitoring system based on the truth data set and the sensor data set.
